(19) United States
(12) Patent Application Publication
(10) Pub. No.: US 2025/0375138 A1
(43) Pub. Date: Dec. 11, 2025

(54) BIOLOGICAL SENSOR

(71) Applicant: NITTO DENKO CORPORATION, Ibaraki-shi, Osaka (JP)

(72) Inventor: Sota KONDO, Ibaraki-shi, Osaka (JP)

(21) Appl. No.: 18/876,518

(22) PCT Filed: Jun. 19, 2023
(86) PCT No.: PCT/JP2023/022611

§ 371 Date: Dec. 18, 2024

(30) Foreign Application Priority Data
Jun. 24, 2022 (JP) 2022-102032

(51) Int. Cl. A61B 5/257; A61B 5/00

(52) U.S. Cl. CPC A61B 5/257; A61B 5/6833; A61B 2562/164

(57) ABSTRACT

A biological sensor according to the present invention includes a sensor body configured to obtain biological information; an electrode connected to the sensor body; a first layer member including a cover member that includes a housing space in which the sensor body is housed, the electrode being disposed on a lower surface of the first layer member; and a second layer member that is attached to the lower surface of the first layer member so as to expose the electrode and cover the sensor body. A tensile modulus of the cover member is 1.5 MPa or less and a relative dielectric constant of the cover member is 2.2 or less.

### TECHNICAL FIELD

The present invention relates to a biological sensor.

### BACKGROUND ART

A biological sensor configured to perform measurement of biological information, such as an electrocardiogram waveform, a pulse wave, an electroencephalogram, an electromyogram, or the like, is used in medical institutions, such as a hospital, a clinic, and the like, nursing facilities, ones' homes, and the like. The biological sensor includes a biological electrode configured to obtain biological information of subjects by contact with their living body. When measuring such biological information, the biological sensor is attached to skin of a subject, and an electric signal of the biological information is obtained by the biological electrode. As a result, measurement of the biological information is performed.

As such a biological sensor, for example, a biological sensor including a sensor body, an electrode, a first layer member, and a second layer member is disclosed. In this biological sensor, the first layer member is formed by stacking a cover on an upper sheet and is configured to house the sensor body, and the second layer member is attached to a surface of the first layer member on the living body side and is formed such that the sensor body is disposed and the electrode is exposed (see, for example, PTL 1).

This biological sensor obtains biological information by attaching, to skin, a first adhesive layer provided on a surface of the first layer member facing the living body and a second adhesive layer provided on a surface of the second layer member facing the living body, and contacting the electrode attached to the first adhesive layer with the skin.

### CITATION LIST

### Patent Literature

PTL 1: Japanese Patent No. 6947955

### SUMMARY OF THE INVENTION

### Technical Problem

Here, when a biological sensor readily deforms and is flexible, the noise detected at the time of obtaining the biological information increases. Therefore, a material having a high hardness, such as silicone rubber or the like, is used as the material of the cover. When the cover is formed to be hard, the deformation of the biological sensor is suppressed, and thus the generation of noise is suppressed. However, the flexibility of the biological sensor is lowered, and it becomes challenging for the biological sensor to deform following the deformation of the surface of the living body. As a result, there is a possibility that an attachment performance to the surface of the living body, such as, for example, skin of a subject, is degraded and likely to cause peeling.

A biological sensor is often used for a long time in a state of being attached to the surface of the living body of a subject, such as skin or the like. Therefore, in order to stably obtain an electric signal indicating biological information for a long time, it is desirable that the biological sensor can be maintained in a state of being stably attached to the surface of the living body while suppressing generation of noise of the detected electric signal.

In one aspect of the present invention, it is an object to provide a biological sensor that can suppress the generation of noise during use and can be stably attached to the living body.

### Solution to the Problem

One aspect of the biological sensor according to the present invention includes: a sensor body configured to obtain biological information; an electrode connected to the sensor body; a first layer member including a cover member that includes a housing space in which the sensor body is housed, the electrode being disposed on a lower surface of the first layer member; and a second layer member that is attached to the lower surface of the first layer member so as to expose the electrode and cover the sensor body. A tensile modulus of the cover member is 1.5 MPa or less and a relative dielectric constant of the cover member is 2.2 or less.

### Advantageous Effects of the Invention

According to one aspect of the present invention, the biological sensor can suppress the generation of noise during use and can be stably attached to the living body.

### BRIEF DESCRIPTION OF THE DRAWINGS

FIG. 1 is a perspective view illustrating an entire configuration of a biological sensor according to an embodiment of the present invention.

FIG. 2 is a plan view illustrating examples of parts of the biological sensor.

FIG. 3 is a longitudinal cross-sectional view of the biological sensor taken along the line I-I in FIG. 1.

FIG. 4 is an explanatory view illustrating the biological sensor of FIG. 1 attached to the chest of a living body.

FIG. 5 is an example of an electrocardiogram waveform without noise.

### DESCRIPTION OF THE EMBODIMENTS

In the following, embodiments of the present invention will be described in detail. For ease of understanding to the description, the same components in the drawings are denoted by the same symbols, and duplicate description is omitted. Also, the scale of the members in the drawings may differ from the actual scale. In this specification, the expression indicating a numerical range: “from . . . through . . . ” means that the numerical value described after “from” and the numerical value described after “through” are included in that numerical range as a lower limit and an upper limit, unless otherwise specified.

### Biological Sensor

A biological sensor according to the present embodiment will be described. The living body refers to, for example, a human body (human) and animals, such as cattle, horses, pigs, chickens, dogs, cats, and the like. The biological sensor according to the present embodiment is suitably used for the living body, especially for a human body. The present embodiment will be described taking, as an example, a case in which the living body is of a human.

The biological sensor according to the present embodiment is an attachment-type biological sensor configured to be attached to a part of a living body (e.g., skin, scalp, forehead, or the like), thereby performing measurement of biological information. In the present embodiment, a description will be given of a case in which the biological sensor is attached to the skin of a human and measures an electric signal (biological signal) indicating biological information of the human.

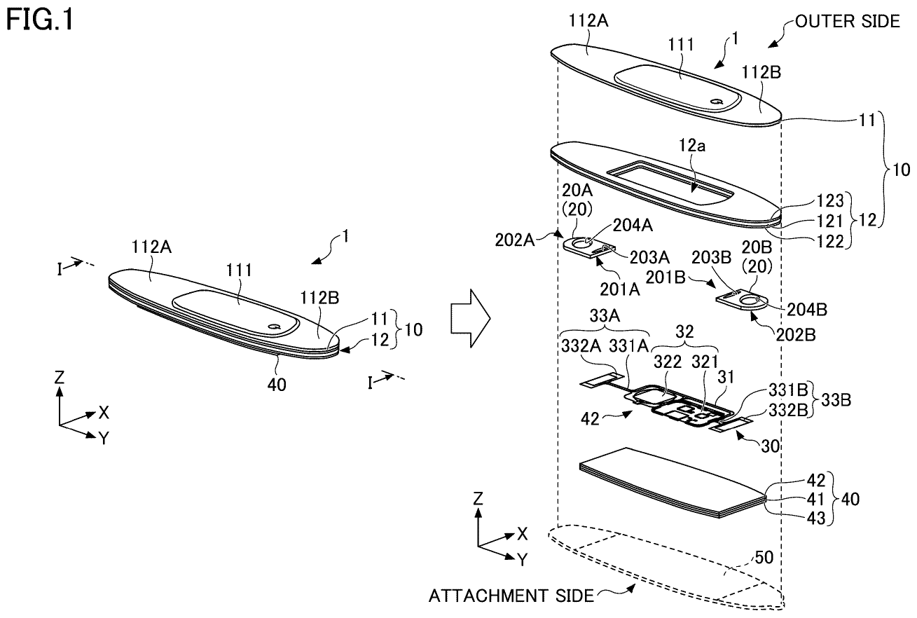

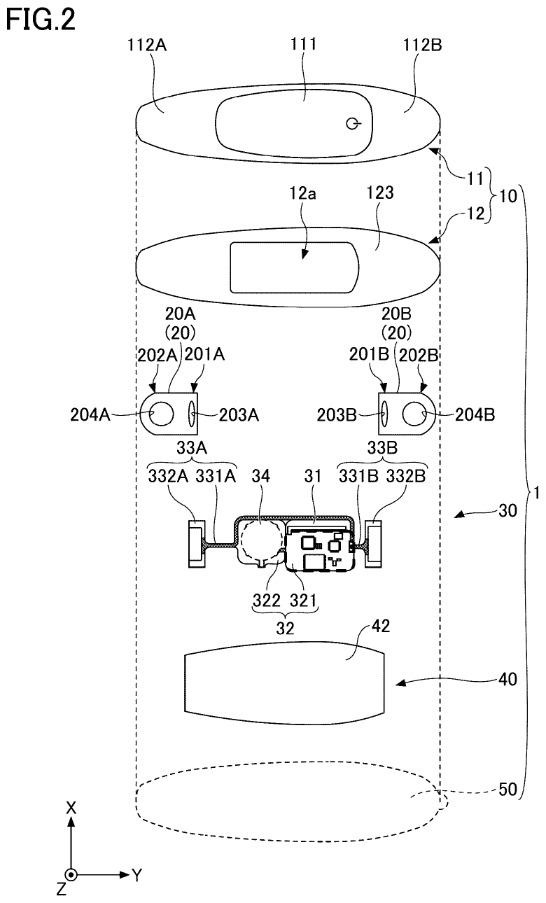

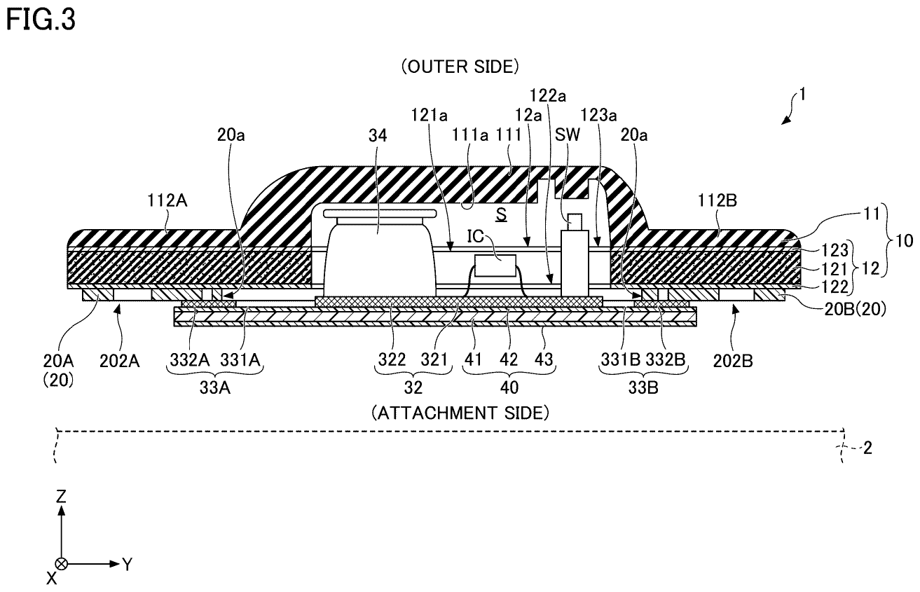

FIG. 1 is a perspective view illustrating the entire configuration of the biological sensor according to the present embodiment. The left-hand view of FIG. 1 illustrates the external appearance of the biological sensor according to the present embodiment, and the right-hand view of FIG. 1 illustrates a state in which the parts of the biological sensor according to the present embodiment are exploded. FIG. 2 is a plan view illustrating examples of the parts of the biological sensor. FIG. 3 is a longitudinal cross-sectional view of the biological sensor taken along the line I-I in FIG. 1.

As illustrated in FIGS. 1 and 2, a biological sensor 1 is a plate-like (sheet-like) member formed in a substantially elliptical shape in a plan view. As illustrated in FIGS. 2 and 3, the biological sensor 1 includes a first layer member 10, an electrode 20, a sensor portion 30, and a second layer member 40, and is formed by stacking the first layer member 10, the electrode 20, and the second layer member 40 in this order from the first layer member 10 side toward the second layer member 40 side. According to the biological sensor 1, the first layer member 10, the electrode 20, and the second layer member 40 form an attachment surface to be attached to a skin 2, which is an example of the living body. The biological sensor 1 attaches the attachment surface to the skin 2 and measures a potential difference (polarization voltage) between the skin 2 and the electrode 20, thereby measuring an electric signal (biological signal) indicating biological information of a subject.

In FIGS. 1 to 3, using a three-dimensional orthogonal coordinate system having three axis directions (X-axis direction, Y-axis direction, and Z-axis direction), the transverse direction of the biological sensor is an X-axis direction, the longitudinal direction of the biological sensor is a Y-axis direction, and the height direction (thickness direction) of the biological sensor is a Z-axis direction. The side (outer side) opposite to the side on which the biological sensor 1 is attached to the living body (subject) (attachment side) is referred to as a +Z-axis direction, and the attachment side is referred to as a −Z-axis direction. In the following description, for the sake of convenience, the +Z-axis direction may be referred to as an upper side or above, and the −Z-axis direction may be referred to as a lower side or below. However, this does not represent a universal vertical relationship.

The biological signal is, for example, an electric signal indicating an electrocardiogram waveform, an electroencephalogram, a pulse, or the like.

In use of the biological sensor 1, the inventors of the present application focused on the flexibility and the amount of charge of a cover member 11 provided on a front-surface side of the first layer member 10. The inventors of the present application have found that, by lowering the tensile modulus and the relative dielectric constant of the cover member 11, the adhesion state of the electrode 20 to the surface of the living body can be maintained and the adhesiveness to the surface of the living body can be enhanced, thereby suppressing generation of noise detected during use of the biological sensor 1 and enhancing an attachment performance of the biological sensor 1 to the living body.

### [First Layer Member]

As illustrated in FIGS. 1 and 2, the first layer member 10 includes the cover member 11 and an upper sheet 12 that are stacked in this order. The cover member 11 and the upper sheet 12 have substantially the same outer shape in a plan view.

### (Cover Member)

As illustrated in FIG. 3, the cover member 11 is positioned on the outermost side (+Z-axis direction) of the biological sensor 1, and is adhered to the upper surface of the upper sheet 12. The cover member 11 includes: a projection 111 that projects in a substantially dome shape in the height direction (+Z-axis direction) in FIG. 1, the projection 111 being in a center region in the longitudinal direction (Y-axis direction); and flat portions 112A and 112B provided at both ends of the cover member 11 in the longitudinal direction (Y-axis direction). The upper and lower surfaces of the projection 111, and the upper and lower surfaces of the flat portions 112A and 112B are formed to be flat.

The cover member 11 has an opening on the inner side (attachment side) of the projection 111 so as to have a recess 111a formed in a recessed shape on the skin 2 side. The recess 111a only needs to have a size sufficient to house at least a part of the sensor portion 30. A housing space S in which the sensor portion 30 is housed is formed, on the inner side (attachment side) of the projection 111, by the recess 111a at the inner surface of the projection 111, the electrode 20, and the second layer member 40.

As a material forming the cover member 11, a flexible material, such as a thermoplastic elastomer or the like, can be used. The cover member 11 formed using the flexible material or the like protects the sensor portion 30 disposed in the housing space S of the cover member 11, and absorbs an impact applied to the biological sensor 1 from the upper surface side to reduce the impact applied to the sensor portion 30.

Examples of the thermoplastic elastomer include polystyrene-based thermoplastic elastomers, polyolefin-based thermoplastic elastomers, polyester-based thermoplastic elastomers, urethane-based thermoplastic elastomers, polyvinyl chloride-based thermoplastic elastomers, polyamide-based thermoplastic elastomers, nitrile-based thermoplastic elastomers, nylon-based thermoplastic elastomers, fluororubber-based thermoplastic elastomers, polybutadiene-based thermoplastic elastomers, ethylene vinyl acetate-based thermoplastic elastomers, chlorinated polyethylene-based thermoplastic elastomers, styrene-butadiene block copolymers or hydrogenated products of the styrene-butadiene block copolymers, styrene-isoprene block copolymers or hydrogenated products of the styrene-isoprene block copolymers, and the like. These may be used alone or in combination. Of these, styrene-based thermoplastic elastomers and polyester-based thermoplastic elastomers are preferable, and styrene-based thermoplastic elastomers are more preferable.

No particular limitation is imposed on the styrene-based thermoplastic elastomer as long as it is a thermoplastic elastomer having a styrene unit (preferably a styrene block unit). Examples of the styrene-based thermoplastic elastomer include styrene-isobutylene-styrene block copolymers (SIBS), styrene-isoprene-styrene block copolymers (SIS), styrene-isobutylene block copolymers (SIB), styrene-butadiene-styrene block copolymers (SBS), styrene-ethylene-butene-styrene block copolymers (SEBS), styrene-ethylene-propylene-styrene block copolymers (SEPS), styrene-ethylene-ethylene-propylene-styrene block copolymers (SEEPS), styrene-butadiene-butylene-styrene block copolymers (SBBS), and the like. These may be used alone or in combination. Of these, SIS, SBS, SEBS, and SBBS are preferable, and SIS and SEBS are more preferable.

As the thermoplastic elastomer, for example, it is possible to use thermoplastic elastomers that are produced or sold by RIKEN TECHNOS CORP., ARONKASEI CO., LTD., DU PONT-TORAY CO., LTD., KANEKA CORPORATION, CLAYTON POLYMERS LTD., Asahi Kasei Corporation, and the like.

The cover member 11 is typically formed using crosslinked rubber or a thermosetting resin, such as an epoxy resin, a phenolic resin, a polyimide resin, an unsaturated polyester resin, a diallyl phthalate resin, or the like. Examples of the crosslinked rubber include natural rubber, acrylic rubber, butadiene rubber, isoprene rubber, styrene-butadiene copolymer rubber, nitrile rubber, hydrogenated nitrile rubber, chloroprene rubber, ethylene-propylene copolymer rubber, chlorinated polyethylene rubber, chlorosulfonated polyethylene rubber, butyl rubber, halogenated butyl rubber, fluororubber, urethane rubber, silicone rubber, and the like. When the cover member 11 is formed of a thermosetting resin, the hardness of the cover member 11 can be increased. Thus, the waveform accuracy of the biological signal measured at the time of the measurement of the biological signal can be suppressed, but it is unlikely to obtain a sufficient attachment performance to the surface of the living body.

When the cover member 11 is an elastomer molded body of the above-described thermoplastic elastomer, the elastomer molded body may be a non-porous elastomer molded body or may be a porous elastomer molded body. However, the elastomer molded body is preferably a porous elastomer molded body. The porous elastomer molded body may be a closed cell foamed elastomer molded body (a porous elastomer molded body produced through foam molding that forms closed cells) or may be a communicating cells foamed elastomer molded body (a porous elastomer molded body produced through foam molding that forms communicating cells).

The thickness of the upper surface and the side walls of the projection 111 may be larger than that of the flat portions 112A and 112B. Thus, the flexibility of the projection 111 can be lower than that of the flat portions 112A and 112B, and the sensor portion 30 can be protected from an external force applied to the biological sensor 1.

The thickness of the upper surface and the side walls of the projection 111 can be appropriately designed and may be, for example, from 1.5 mm through 3 mm. The thickness of the flat portions 112A and 112B can also be appropriately designed and may be, for example, from 0.5 mm through 1 mm.

The flat portions 112A and 112B, which are thinner, have higher flexibility than that of the projection 111. Thus, when the biological sensor 1 is attached to the skin 2, they readily deform in accordance with deformation of the surface of the skin 2 caused by body movements, such as extension, bending, twisting, and the like. This can reduce stress applied to the flat portions 112A and 112B in response to deformation of the surface of the skin 2, and can suppress peeling of the biological sensor 1 off from the skin 2.

The outer peripheral portions of the flat portions 112A and 112B may have a shape in which the thickness gradually decreases toward the respective ends. This can further increase the flexibility of the outer peripheral portions of the flat portions 112A and 112B, and can improve sensation during attachment of the biological sensor 1 to the skin 2 compared to a case in which the thickness of the outer peripheral portions of the flat portions 112A and 112B are not made smaller.

The hardness of the cover member 11 can be appropriately designed to have a desirable magnitude. For example, the hardness of the cover member 11 is preferably from 10 through 40. The upper limit of the hardness is further preferably 30 or less. When the hardness of the cover member 11 is within the above preferable range, the upper sheet 12, the electrode 20, and the second layer member 40 can readily deform in accordance with the movement of the skin 2 without being influenced by the cover member 11 when the skin 2 is extended by the body movements. The hardness (how hard it is) refers to Shore A hardness. In the present specification, the Shore A hardness refers to a hardness as measured in accordance with ISO7619 (JIS K 6253-3:2012). As described in “Rubber, vulcanized or thermoplastic-Determination of hardness Part 3: Durometer method” of JIS K 6253-3:2012, the measured value of Shore A hardness measured by preparing a sheet sample of the cover member 11 can be used as the Shore A hardness of the cover member 11.

The tensile modulus of the cover member 11 is preferably 1.5 MPa or less, more preferably 1.2 MPa or less, and further preferably 1.0 MPa or less, for example, at normal temperature (23° C.±2° C.). When the tensile modulus of the cover member 11 is too high, the cover member 11 becomes harder and is unlikely to stretch. When the tensile modulus of the cover member 11 is too low, the cover member 11 readily stretches, but noise is likely to occur during use. Therefore, for example, the tensile modulus of the cover member 11 may be 1.5 MPa or less. When the tensile modulus of the electrode 20 at normal temperature (23° C.±2° C.) is 1.5 MPa or less, the cover member 11 can relax the stress generated by the deformation of the surface of the living body, and can exhibit excellent stretchability with respect to the surface of the skin 2.

The tensile modulus of the cover member 11 can be measured by a method in accordance with JIS K7161: 2014 or the like. When the tensile modulus of the cover member 11 is determined in accordance with JIS K7161: 2014, the cover member 11 is cut to prepare a rectangular (e.g., 30 mm in length×10 mm in width) test piece (sample) having a predetermined size. Both ends of the test piece are held between chucks such that the distance between the chucks is 20 mm. In this state, a tensile test is performed under conditions in which the temperature is normal temperature (23° C.) and the tensile speed is 30 mm/min, thereby obtaining a stress-strain curve. The tensile modulus at normal temperature (23° C.±2° C.) can be calculated in accordance with the obtained stress-strain curve, i.e., by obtaining the slopes of the curve at two points at which the strain is 0.05% and 0.25%. Specifically, a tensile modulus E (unit: MPa) at normal temperature (23° C.) can be determined by dividing the difference in stress (σ2−σ1) by the difference in strain (ε2−ε1), as presented in Formula (1) below:

$$E = ( σ2 - σ1 ) / ( ε2 - ε1 ) , \qquad ( 1 )$$

where ε1 denotes a value when the strain (unit: %) of the cover member 11 is 0.05%, ε2 denotes a value when the strain (unit: %) of the cover member 11 is 0.25%, σ1 denotes the stress (unit: MPa) corresponding to ε1, and σ2 denotes the stress (unit: MPa) corresponding to ε2.

The relative dielectric constant of the cover member 11 is 2.2 or less, preferably 2.0 or less, and more preferably 1.8 or less. When the relative dielectric constant of the cover member 11 is 2.2 or less, the impedance is increased and the generation of noise is suppressed.

The relative dielectric constant of the cover member 11 can be determined by a typical measurement method for the relative dielectric constant. For example, the cover member 11 is cut to prepare a circular (e.g., 38 mm or greater in diameter) test piece (sample) having a predetermined size. A capacitance (electric capacitance) C of the test piece is measured using an impedance analyzer. The relative dielectric constant, ε/ε₀, can be calculated from the measured capacitance C. Specifically, the relative dielectric constant (ε/ε₀) of the cover member 11 can be calculated by dividing a value C×d, the product of the capacitance C of the test piece and the diameter d of the test piece, by the area S of the test piece and the square of the dielectric constant in vacuum, ε₀, (≈8.85×10⁻¹² F/m), i.e., (C×d/(S×ε₀²)). The relative dielectric constant of the cover member 11 is generally dependent on the frequency. The biological sensor 1 is used for the measurement of an electrocardiogram, and the frequency of an electrocardiogram is mainly from several hertz through 30 Hz. Therefore, the relative dielectric constant at the time of calculation of the relative dielectric constant can be 5 Hz.

The amount of charge of the cover member 11 is preferably from −1.0 kV through 1.0 kV, and more preferably from −0.5 kV through 0.5 kV. When the amount of charge of the cover member 11 is within the above range, the cover member 11 can suppress charging that may occur through rubbing against the upper sheet 12 or the like due to deformation of the biological sensor. The amount of charge of the cover member 11 can be measured using a typical digital electrostatic potential meter. The measurement may be performed at an environmental temperature of about 23° C.±2° C. and at an environmental humidity of about 40%. Alternatively, a carrier film (available from Nitto Denko Corporation, product name “E-MASK”) may be attached to the cover member 11 for removal of charges, and then the amount of charge of the cover member 11 may be measured.

The adhesive strength of the cover member 11 with respect to an adhesive may be equal to or less than a desirable magnitude in accordance with the type of the adhesive. When the adhesive is a silicone resin or an acrylic resin, the adhesive strength of the cover member 11 with respect to a silicone resin or an acrylic resin may be 1.7 N/10 mm or more. The adhesive strength of the cover member 11 with respect to a silicone resin or an acrylic resin is a peeling strength of the cover member 11 from the silicone resin or the acrylic resin. When the adhesive strength of the cover member 11 is equal to or less than the above adhesive strength, the cover member 11 exhibits good adhesiveness with respect to the upper sheet 12. In addition, advantageously, the cover member 11 is readily attached to the upper sheet 12 through typical pressure bonding.

The adhesive strength of the cover member 11 with respect to the adhesive can be measured in accordance with JIS Z 0237:2000. For example, the cover member 11 is cut into a predetermined size (e.g., 10 mm wide and 100 mm long) to prepare a test piece. One surface of the test piece is pressure-bonded to a PET film that has a predetermined thickness and to which an adhesive is attached, thereby preparing a measurement sample. The pressure-bonding may be performed under pressure-bonding conditions in which a roller of 2 kg is caused to go back and forth.

After the test piece is pressure-bonded to the PET film, aging may be performed in an atmosphere of 23° C. and 50% RH for several minutes (e.g., 30 minutes). As the adhesive, double-sided adhesive tape or the like having an adhesive layer formed of a silicone resin or an acrylic resin may be used. The adhesive layer preferably has high adhesiveness. As the double-sided adhesive tape having an adhesive layer formed of a silicone resin, specifically, ST503 (available from Nitto Denko Corporation) or the like may be used. As the double-sided adhesive tape having an adhesive layer formed of an acrylic resin, specifically, PKE-20 (available from Nitto Denko Corporation) or the like may be used. After aging, in an atmosphere of 25° C. and 50% RH in accordance with JIS Z 0237, the measurement sample is peeled off from the PET film using a tensile tester at a tensile speed of 300 mm/min and a peeling angle of 180°,thereby measuring an adhesive strength at a peeling angle of 180° (unit: N/10 mm). As the tensile tester, for example, “Precision Universal Tester, Autograph AG-IS 50 N”, available from Shimadzu Corporation, may be used.

### (Upper Sheet)

As illustrated in FIG. 3, the upper sheet 12 is adhered to the lower surface of the cover member 11. The upper sheet 12 has a through-hole 12a at a position facing the projection 111 of the cover member 11. Owing to the through-hole 12a, a sensor body 32 of the sensor portion 30 can be housed in the housing space S, formed by the recess 111a at the inner surface of the cover member 11 and the through-hole 12a, without being blocked by the upper sheet 12.

The upper sheet 12 includes: a first base 121; a first adhesive layer 122 that is provided at one surface of the first base 121 facing the electrode 20 and to which the electrode 20 is attached; and an upper adhesive layer 123 that is provided at the surface of the first base 121 opposite to the surface facing the electrode 20.

### ((First Base))

As illustrated in FIG. 3, the first base 121 is provided on the attachment side that is the opening side of the cover member 11. As illustrated in FIG. 1, The first base 121 is formed in a sheet shape. The first base 121 may be formed of a porous body having a porous structure and having flexibility, waterproofness, and moisture permeability. As the porous body, for example, a foamed material (foamed body) having cells, such as open cells, closed cells, and semi-closed cells, can be used. As such, water vapor derived from sweat or the like generated from the skin 2, to which the biological sensor 1 is attached, can be released to the exterior of the biological sensor 1 through the first base 121.

The moisture permeability of the first base 121 is preferably from 100 g/(m²·day) through 5,000 g/(m²·day). By setting the moisture permeability of the first base 121 to be in the range of from 100 g/(m²·day) through 5,000 g/(m²·day), the water vapor entering the first base 121 from one surface can pass through the first base 121, and can be stably released from the other surface.

As the material forming the first base 121, a thermoplastic resin can be used, and examples of the thermoplastic resin include polyurethane-based resins, polystyrene-based resins, polyolefin-based resins, silicone-based resins, acrylic resins, vinyl chloride-based resins, polyester-based resins, and the like. As the first base 121, for example, FOLEC available from INOAC CORPORATION may be used.

The thickness of the first base 121 may be appropriately set, and, for example, may be from 0.5 mm through 1.5 mm.

The first base 121 has a through-hole 121a at a position facing the projection 111 of the cover member 11. When the first adhesive layer 122 and the upper adhesive layer 123 are provided on the surface of the first base 121 other than the through-hole 121a, through-holes 122a and 123a can also be formed in the first adhesive layer 122 and the upper adhesive layer 123. The through-holes 121a, 122a, and 123a form the through-hole 12a.

The first base 121 may be a base having no porous structure as long as the base has flexibility, waterproofness, and moisture permeability. Because the first base 121 has flexibility, waterproofness, and moisture permeability, the first base 121 can be readily stretched in the state of contacting the skin 2. Thus, the state of contacting the skin 2 can be maintained, and also the entry of liquid into the gap between the first base 121 and the upper adhesive layer 123 can be suppressed. Further, water vapor derived from sweat or the like generated from the skin 2, to which the biological sensor 1 is attached, can be released to the exterior of the biological sensor 1 through the first base 121. Therefore, the upper sheet 12 readily maintains adhesion durability.

As the material of the base material having no porous structure, a thermoplastic resin can be used similar to the above, and examples of the thermoplastic resin include polyurethane-based resins, polystyrene-based resins, polyolefin-based resins, silicone-based resins, acrylic resins, vinyl chloride-based resins, polyester-based resins, and the like. When the first base 121 is formed of a base material having no porous structure, a polyurethane sheet, such as, for example, ESMER URS available from Nihon Matai Co., Ltd., can be used as the first base 121.

((First Adhesive layer))

As illustrated in FIG. 3, the first adhesive layer 122 is attached to one surface of the first base 121 facing the electrode 20. The first adhesive layer 122 is positioned at a surface of the first base 121 facing the living body (−Z-axis direction), and has the function of adhering the skin 2 and the first base 121 to each other, the function of adhering the first base 121 and a second base 41 to each other, and the function of adhering the first base 121 and the electrode 20 to each other.

The first adhesive layer 122 may have moisture permeability. As such, as described below, water vapor derived from sweat or the like generated from the skin 2, to which the biological sensor 1 is attached, can be escaped to the first base 121 through the first adhesive layer 122, and can be released to the exterior of the biological sensor 1 through the first base 121. When the first base 121 has a cell structure as described above, water vapor can be released to the exterior of the biological sensor 1 through the first adhesive layer 122. This can prevent sweat or water vapor from accumulating at the interface between the skin 2, on which the biological sensor 1 is attached, and the first layer member 10. As a result, it is possible to prevent the adhesive strength of the first adhesive layer 122 from weakening due to the moisture accumulated at the interface between the skin 2 and the first adhesive layer 122, and prevent peeling of the biological sensor 1 off from the skin 2.

Preferably, the moisture permeability of the first adhesive layer 122 is, for example, 1 g/(m²·day) or more. The moisture permeability of the first adhesive layer 122 may be 10,000 g/(m²·day) or less. As long as the moisture permeability of the first adhesive layer 122 is 1 g/(m²·day) or more, when the first adhesive layer 122 is attached to the skin 2, sweat or the like delivered from the first adhesive layer 122 can be released toward the exterior. This can reduce the burden on the skin 2.

The material forming the first adhesive layer 122 is preferably a material having pressure-sensitive adhesiveness. For example, an acrylic pressure-sensitive adhesive may be used.

The first adhesive layer 122 may be adhesive tape formed of the above material. When the cover member 11 is stacked over the first adhesive layer 122 to form the biological sensor 1, it is possible to enhance waterproofness of the biological sensor 1, and enhance the bonding strength with the cover member 11.

A wavy pattern (web pattern) may be formed on the surface of the first adhesive layer 122. This wavy pattern is formed by repeatedly and alternatingly arranging recesses for a thickness smaller than that of the other portions (or for zero thickness). As the first adhesive layer 122, for example, adhesive tape having a web pattern formed on a surface of the adhesive tape may be used. The first adhesive layer 122 has a web pattern on the surface, and as a result, the surface of the first adhesive layer 122 includes both of: portions in which an adhesive is likely to contact the living body; and portions in which the adhesive is unlikely to contact the living body. Because the surface of the first adhesive layer 122 includes both of the portions in which the adhesive is present and the portions in which the adhesive is absent, the portions that are likely to contact the living body can be sparsely located on the surface of the first adhesive layer 122. The moisture permeability of the first adhesive layer 122 tends to increase as the adhesive is thinner. Therefore, by forming the web pattern on the surface of the first adhesive layer 122 and providing the surface of the first adhesive layer 122 with portions in which the adhesive is thinner, it is possible to enhance the moisture permeability while maintaining the adhesive strength, compared to a case in which the web pattern is not formed. The shape of the recess may be a straight shape or a circular shape, in addition to a wavy shape.

The thickness of the first adhesive layer 122 may be desirably set, and is preferably from 10 μm through 300 μm, more preferably from 50 μm through 200 μm, and further preferably from 70 μm through 110 μm. When the thickness of the first adhesive layer 122 is from 10 μm through 300 μm, the biological sensor 1 can be reduced in thickness.

### ((Upper Adhesive Layer))

As illustrated in FIG. 3, the upper adhesive layer 123 is attached to the surface of the first base 121 opposite to the surface facing the electrode 20. The upper adhesive layer 123 is attached to the upper surface of the first base 121 and at a position corresponding to the flat surface on the attachment side (−Z-axis direction) of the cover member 11. The upper adhesive layer 123 has the function of adhering the first base 121 and the cover member 11 to each other.

As the material forming the upper adhesive layer 123, a silicone-based adhesive, silicone tape, or the like, can be used.

The thickness of the upper adhesive layer 123 may be appropriately set, and, for example, may be from 10 μm through 300 μm.

### [Electrode]

As illustrated in FIG. 3, the electrode 20 is attached to the lower surface of the first adhesive layer 122 on the attachment side (−Z-axis direction) in a state in which a part of the electrode 20 on the sensor body 32 side is connected to interconnects 331A and 331B and is held between the first adhesive layer 122 and a lower adhesive layer 42. The electrode 20 contacts the living body at a portion that is not held between the first adhesive layer 122 and the lower adhesive layer 42. When the biological sensor 1 is attached to the skin 2, the electrode 20 contacts the skin 2, thereby enabling detecting biological signals. The electrode 20 may be embedded in the second base 41 in a state in which the electrode 20 is exposed so as to be able to contact the skin 2.

The electrode 20 is formed by a pair of electrodes 20A and 20B. As illustrated in FIG. 3, the electrode 20A is disposed on the left-hand side in the drawing, and the electrode 20B is disposed on the right-hand side in the drawing. One end (inner side) of the electrode 20A in the longitudinal direction (Y-axis direction) contacts a terminal 332A, and one end (inner side) of the electrode 20B in the longitudinal direction (Y-axis direction) contacts a terminal 332B. The pair of electrodes 20A and 20B have substantially the same shape.

The one end of the electrode 20A that contacts the terminal 332A of the sensor portion 30 is referred to as a facing portion 201A, and the one end of the electrode 20B that contacts the terminal 332B of the sensor portion 30 is referred to as a facing portion 201B. A portion of the electrode 20A that does not contact the terminal 332A (the other end (outer side) in the longitudinal direction (Y-axis direction)) is referred to as an exposed portion 202A, and a portion of the electrode 20B that does not contact the terminal 332B (the other end (outer side) in the longitudinal direction (Y-axis direction)) is referred to as an exposed portion 202B.

The electrode 20 may have any shape, such as a sheet shape or the like.

No particular limitation is imposed on the shape of the electrode 20 in a plan view. The electrode 20 may be designed to have a shape that is appropriate in accordance with applications or the like. As illustrated in FIG. 2, the electrode 20A or 20B may be formed such that in a plan view, the one end, i.e., the facing portion 201A or 201B, is formed in a rectangular shape, and the other end, i.e., the exposed portion 202A or 202B, is formed in an arc shape.

As illustrated in FIGS. 2 and 3, the electrode 20A or 20B is provided at the one end (inner side) in the longitudinal direction (Y-axis direction), and may have: a through-hole 203A or 203B formed at the one end (inner side) and having an oval shape that is thin and long in the width direction (X-axis direction); and a through-hole 204A or 204B formed to be circular at the other end (outer side) in the longitudinal direction (Y-axis direction). Thus, the electrode 20 can expose the first adhesive layer 122 from the through-holes 203A and 203B and the through-holes 204A and 204B to the attachment side in a state of being attached to the first adhesive layer 122, thereby enhancing adhesiveness between the electrode 20 and the skin 2. No particular limitation is imposed on the number of the through-holes 203A and 203B or the through-holes 204A and 204B. The number of the through-holes 203A and 203B or the through-holes 204A and 204B may be appropriately set in accordance with the sizes and the like of the facing portions 201A and 201B of the electrode 20.

The electrode 20 can be formed using a cured product of a conductive composition containing a conductive polymer and a binder resin, a metal, an alloy, or the like. Of these, it is preferable to form the electrode 20 using a cured product of a conductive composition from the viewpoint of safety of the living body, such as, for example, avoiding an allergic reaction or the like occurring when the electrode 20 is applied to the living body. The electrode 20 for use may be an adhesive electrode sheet that is obtained by forming a cured product of a conductive composition in the form of a sheet.

As the conductive polymer, for example, it is possible to use a polythiophene-based conductive polymer, a polyaniline-based conductive polymer, a polyacetylene-based conductive polymer, a polypyrrole-based conductive polymer, a polyphenylene-based conductive polymer, a derivative of the above-listed polymers, a composite of the above-listed polymers, or the like. Of these, it is preferable to use composites in which polythiophene is doped with polyaniline as a dopant. Of the composites of polythiophene and polyaniline, it is more preferable to use PEDOT/PSS in which poly(3,4-ethylenedioxythiophene) (also referred to as PEDOT), which is polythiophene, is doped with polystyrene sulfonic acid (poly 4-styrenesulfonate; PSS), which is polyaniline. This is because of low contact impedance with the living body and high conductivity.

The binder resin for use may be a water-soluble polymer, a water-insoluble polymer, or the like. The water-soluble polymer for use may be a hydroxyl group-containing polymer, such as polyvinyl alcohol (PVA), modified PVA, and the like.

The conductive composition may appropriately contain various typical additives, such as a crosslinking agent, a plasticizer, and the like, in a desired ratio. Examples of the crosslinking agent include aldehyde compounds, such as sodium glyoxylate and the like. Examples of the plasticizer include glycerin, ethylene glycol, propylene glycol, and the like.

The metal and the alloy for use may be typical metals and alloys, such as Au, Pt, Ag, Cu, Al, and the like.

The thickness of the electrode 20 may be an appropriate height and may be, for example, from 10 μm through 100 μm. When the thickness of the electrode 20 is within the above preferable range, the electrode 20 can have sufficient strength and flexibility.

The thickness of the electrode 20 is a length of the electrode 20 in a direction perpendicular to the surface of the electrode 20. The thickness of the electrode 20 is, for example, a thickness as measured at a given site in a cross section of the electrode 20. When measurement is performed at a plurality of given sites, the average value of the thicknesses measured at the plurality of given sites may be used as the thickness of the electrode 20.

### (Sensor Portion)

As illustrated in FIG. 3, the sensor portion 30 has a flexible substrate 31, a sensor body 32, and connection portions 33A and 33B connected to the sensor body 32.

The flexible substrate 31 is a resin substrate on which various parts configured to obtain biological information are mounted. The sensor body 32 and the connection portions 33A and 33B are disposed on the flexible substrate 31.

As illustrated in FIG. 2, the sensor body 32 includes a part-mounting portion 321, serving as a controller, and a battery-mounting portion 322, and obtains biological information.

The part-mounting portion 321 includes various parts mounted on the flexible substrate 31, and obtains biological information. These parts are: a CPU and an integrated circuit configured to process biological signals obtained from the living body and generate biological signal data; a switch SW configured to start-up the biological sensor 1; a flash memory configured to store biological signals; a light-emitting element; and the like. An example of a circuit formed of these parts is omitted. The part-mounting portion 321 is driven by power supplied from a battery 34 mounted on the battery-mounting portion 322.

The part-mounting portion 321 is configured to perform wired or wireless transmission to external devices, such as a drive identifier configured to confirm initial driving, a reader configured to read biological information from the biological sensor 1, and the like.

The battery-mounting portion 322 is disposed between the connection portion 33A and the part-mounting portion 321, and is configured to supply power to an integrated circuit or the like mounted on the part-mounting portion 321. As illustrated in FIG. 2, the battery 34 is mounted on the battery-mounting portion 322.

In the longitudinal direction (Y-axis direction) of the sensor body 32, the connection portions 33A and 33B include: the interconnects 331A and 331B connected to the sensor body 32; and the terminals 332A and 332B provided at the distal ends of the interconnects 331A and 331B and connected to the electrode 20.

As illustrated in FIG. 3, one end of the interconnect 331A or 331B is connected to the electrode 20. As illustrated in FIG. 3, the other end of the interconnect 331A is connected to the switch SW or the like mounted on the part-mounting portion 321 along the outer periphery of the sensor body 32. The other end of the interconnect 331B is connected to the switch SW and the like mounted on the part-mounting portion 321.

The terminal 332A or 332B is disposed in a state in which one end of the terminal 332A or 332B is connected to the interconnect 331A or 331B, and the upper surface of the other end of the terminal 332A or 332B is in contact with the electrode 20 and is held between the first layer member 10 and the second layer member 40.

A publicly known battery can be used as the battery 34. For example, a coin-type battery, such as CR2025 or the like, can be used as the battery 34.

### [Second Layer Member]

As illustrated in FIG. 3, the second layer member 40 is provided on an attachment surface side of the electrode 20 and the sensor portion 30. The second layer member 40 is a support substrate on which the sensor portion 30 is provided, and also forms a part of the attachment surface to the skin 2. As illustrated in FIGS. 1 and 2, the outer shape of the second layer member 40 at both sides in the width direction (X-axis direction) may be formed into substantially the same shape as the outer shape of the first layer member 10 at both sides in the width direction (X-axis direction). The length (Y-axis direction) of the second layer member 40 is formed to be shorter than the length (Y-axis direction) of the cover member 11 and the upper sheet 12. As illustrated in FIG. 3, both of the longitudinal ends of the second layer member 40 are located at positions that hold the interconnects 331A and 331B of the sensor portion 30 between the second layer member 40 and the upper sheet 12, and that overlap with a part of the electrode 20.

The second layer member 40 includes the second base 41, the lower adhesive layer 42 provided on the upper surface of the second base 41, and a second adhesive layer 43 provided on the lower surface of the second base 41. The second base 41, the lower adhesive layer 42, and the second adhesive layer 43 may be formed in the same shape in a plan view. The attachment surface to the skin 2 is formed by the second adhesive layer 43 of the second layer member 40 and the electrode 20. In accordance with the area of the electrode 20 and the second adhesive layer 43, the waterproofness and the moisture permeability are different from position to position on the attachment surface, and thus the adhesiveness can be made different. Therefore, it is possible to enable the waterproofness, the moisture permeability, and the adhesiveness to differ in accordance with the area of the attachment surface of the second adhesive layer 43.

### (Second Base)

The second base 41 can be formed of a flexible resin having appropriate stretchability, flexibility, and toughness. As a material forming the second base 41, for example, it is possible to use a thermoplastic resin including: a polyester-based resin, such as polyethylene terephthalate (PET), polybutylene terephthalate, polytrimethylene terephthalate, polyethylene naphthalate, polybutylene naphthalate, or the like; an acrylic resin, such as polyacrylic acid, polymethacrylic acid, polymethyl acrylate, polymethyl methacrylate (PMMA), polyethyl methacrylate, polybutyl acrylate, or the like; a polyolefin-based resin, such as polyethylene, polypropylene, or the like; a polystyrene-based resin, such as polystyrene, an imide-modified polystyrene, an acrylonitrile-butadiene-styrene (ABS) resin, an imide-modified ABS resin, a styrene-acrylonitrile copolymer (SAN) resin, an acrylonitrile ethylene-propylene-diene styrene (AES) resin, or the like; a polyimide-based resin; a polyurethane-based resin; a silicone-based resin; a polyvinyl chloride-based resin, such as polyvinyl chloride, a vinyl chloride-vinyl acetate copolymer resin, or the like. Of these, a polyolefin-based resin and PET are preferably used. These thermoplastic resins have waterproofness that does not permit permeation of water and water vapor (low in water permeability). Therefore, when the second base 41 is formed of any of these thermoplastic resins, it is possible to suppress the entry of sweat or water vapor generated from the skin 2 into the flexible substrate 31 of the sensor portion 30 through the second base 41 in a state in which the biological sensor 1 is attached to the skin 2 of the living body.

The second base 41 is preferably formed in a flat-plate shape because the sensor portion 30 is disposed on the upper surface via the lower adhesive layer 42.

The thickness of the second base 41 can be appropriately selected and, for example, may be from 1 μm through 300 μm.

### (Lower Adhesive Layer)

As illustrated in FIG. 3, the lower adhesive layer 42 is provided on the upper surface of the second base 41 on the cover member 11 side (+Z-axis direction), and the sensor portion 30 is adhered to the lower adhesive layer 42. Both longitudinal ends of the lower adhesive layer 42 of the second layer member 40 are provided at positions that face the facing portions 201A and 201B of the electrode 20. As such, the facing portions 201A and 201B of the electrode 20 and the terminals 332A and 332B can be held between the upper sheet 12 and the second layer member 40 in a state of being compressed, and the electrode 20 and the terminals 332A and 332B can be electrically connected. The lower adhesive layer 42 can be formed of a material similar to that of the second adhesive layer 43 described below, and details will be omitted. The lower adhesive layer 42 does not necessarily need to be provided, and may be absent.

### (Second Adhesive Layer)

As illustrated in FIG. 3, the second adhesive layer 43 is provided on the lower surface of the second base 41 on the attachment side (−Z-axis direction) and contacts the living body.

The second adhesive layer 43 preferably has pressure-sensitive adhesiveness. By virtue of the pressure-sensitive adhesiveness of the second adhesive layer 43, the biological sensor 1 can be readily attached to the skin 2 by pressing the biological sensor 1 against the skin 2 of the living body.

No particular limitation is imposed on the material of the second adhesive layer 43 as long as the material has pressure-sensitive adhesiveness, and the material is a biocompatible material or the like. Examples of the material forming the second adhesive layer 43 include acrylic pressure-sensitive adhesives, silicone-based pressure-sensitive adhesives, and the like. Silicone-based pressure-sensitive adhesives are preferable.

The acrylic pressure-sensitive adhesive preferably contains an acrylic polymer as a main component. The acrylic polymer can function as a pressure-sensitive adhesive component. The acrylic polymer for use is a polymer obtained through polymerization of a monomer component containing a (meth)acrylic acid ester, such as isononyl acrylate, methoxyethyl acrylate, or the like, as a main component and a monomer copolymerizable with a (meth)acrylic acid ester, such as acrylic acid or the like, as an optional component.

The acrylic pressure-sensitive adhesive preferably further contains a carboxylic acid ester. The carboxylic acid ester functions as an adjuster for pressure-sensitive adhesive strength that adjusts the pressure-sensitive adhesive strength of the second adhesive layer 43 by reducing the pressure-sensitive adhesive strength of the acrylic polymer. As the carboxylic acid ester, a carboxylic acid ester compatible with the acrylic polymer can be used. As the carboxylic acid ester, fatty acid triglyceride or the like can be used.

If necessary, the acrylic pressure-sensitive adhesive may contain a crosslinking agent. The crosslinking agent is a crosslinking component that crosslinks the acrylic polymer. Examples of the crosslinking agent include polyisocyanate compounds (polyfunctional isocyanate compounds), epoxy compounds, melamine compounds, peroxide compounds, urea compounds, metal alkoxide compounds, metal chelate compounds, metal salt compounds, carbodiimide compounds, oxazoline compounds, aziridine compounds, amine compounds, and the like. Of these, polyisocyanate compounds are preferable. These crosslinking agents may be used alone or in combination.

The second adhesive layer 43 preferably has excellent biocompatibility. For example, when the second adhesive layer 43 is subjected to a keratin peeling test, a keratin-peeled area percentage is preferably from 0% through 50%. When the keratin-peeled area percentage is in the range of from 0% through 50%, the burden on the skin 2 can be suppressed even if the second adhesive layer 43 is attached to the skin 2.

The second adhesive layer 43 preferably has moisture permeability. Water vapor and the like generated from the skin 2, to which the biological sensor 1 is attached, can be escaped toward the upper sheet 12 through the second adhesive layer 43. Also, as described below, the upper sheet 12 has a structure having cells. Thus, water vapor can be released to the exterior of the biological sensor 1 through the second adhesive layer 43. This can prevent sweat or water vapor from accumulating at the interface between the skin 2, to which the biological sensor 1 is attached, and the second adhesive layer 43. As a result, it is possible to prevent the adhesive strength of the second adhesive layer 43 from weakening due to the moisture accumulated at the interface between the skin 2 and the second adhesive layer 43, and prevent peeling of the biological sensor 1 off from the skin 2.

Preferably, the moisture permeability of the second adhesive layer 43 is, for example, from 300 g/(m²·day) through 10,000 g/(m²·day). When the moisture permeability of the second adhesive layer 43 is in the above preferable range, even if the second adhesive layer 43 is attached to the skin 2, sweat or the like generated from the skin 2 can be appropriately released toward the exterior through the second adhesive layer 43. This can reduce the burden on the skin 2.

The thickness of the second adhesive layer 43 can be appropriately selected, and is preferably from 10 μm through 300 μm. When the thickness of the second adhesive layer 43 is from 10 μm through 300 μm, the biological sensor 1 can become thinner.

As illustrated in FIGS. 1 and 2, when the biological sensor 1 is not in use, a release liner 50 is preferably attached to the surfaces of the electrode 20 and the second base 41 to be attached to the living body until use in order to protect the electrode 20 and the second layer member 40. Upon use, the release liner 50 is peeled off from the electrode 20 and the second layer member 40, and then the attachment surface of the biological sensor 1 can be attached to the skin 2. When the release liner 50 is attached to the attachment surface, the adhesive strength of the electrode 20 and the second layer member 40 can be maintained, for example, even if the biological sensor 1 is stored for a long time. Therefore, by peeling off the release liner 50 from the second layer member 40 and the electrode 20 upon use, the attachment surface can be reliably attached to the skin 2 for use.

No particular limitation is imposed on a production method of the biological sensor 1. However, the biological sensor 1 can be produced by any appropriate method. An example of the production method of the biological sensor 1 will be described.

The first layer member 10, the electrode 20, the sensor portion 30, and the second layer member 40 illustrated in FIGS. 1 and 2 are provided. No particular limitation is imposed on production methods of the first layer member 10, the electrode 20, the sensor portion 30, and the second layer member 40 as long as they can be produced. The first layer member 10, the electrode 20, the sensor portion 30, and the second layer member 40 can be produced by any appropriate methods.

After providing the first layer member 10, the electrode 20, the sensor portion 30, and the second layer member 40 that form the biological sensor 1 illustrated in FIG. 1, the sensor portion 30 is placed on the second layer member 40. Subsequently, the first layer member 10, the electrode 20, the sensor portion 30, and the second layer member 40 are stacked in the order from the first layer member 10 side toward the second layer member 40 side. In this manner, the biological sensor 1 illustrated in FIG. 1 is obtained.

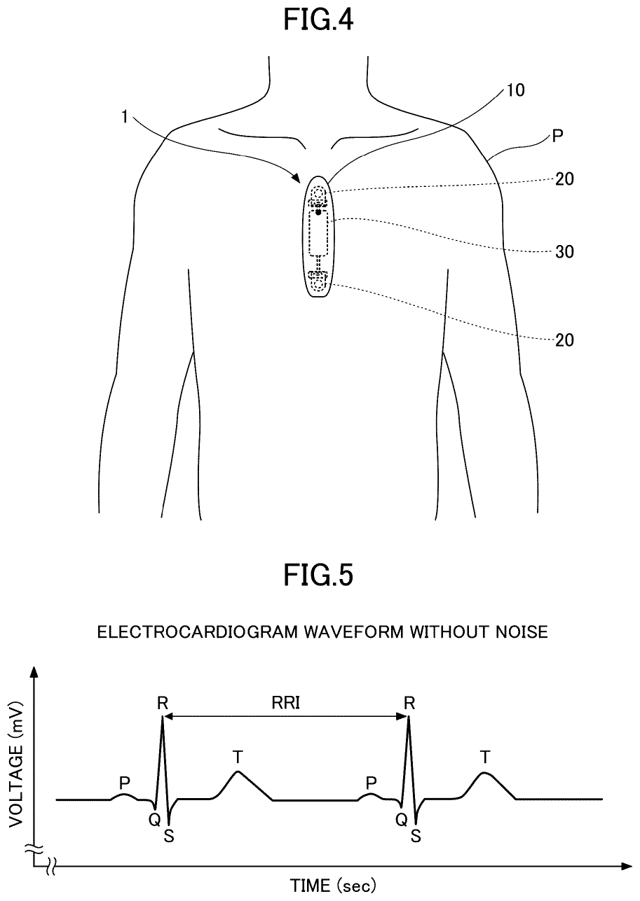

FIG. 4 is an explanatory view illustrating the biological sensor 1 of FIG. 1 attached to the chest of a subject P. As illustrated in FIG. 4, for example, the biological sensor 1 is attached to the skin of the subject P in a state in which the longitudinal direction (Y-axis direction) is aligned with the sternum of the subject P, and one electrode 20 faces upward and the other electrode 20 faces downward. When the biological sensor 1 is attached to the skin of the subject P by the effect of the second adhesive layer 43 of FIG. 2, the biological sensor 1 obtains biological signals, such as an electrocardiogram signal and the like, from the subject P via the electrode 20 in a state in which the electrode 20 is compressed to the skin of the subject P. The biological sensor 1 stores the obtained biological signal data in a non-volatile memory, such as a flash memory or the like, mounted on the part-mounting portion 321.

As described above, the biological sensor 1 includes the first layer member 10, the electrode 20, the sensor body 32, and the second layer member 40, and the tensile modulus and the relative dielectric constant of the cover member 11 included in the first layer member 10 are 1.5 MPa or less and 2.2 or less, respectively. Thus, the cover member 11, which has a tensile modulus of 1.5 MPa or less, becomes flexible, and can be readily extended, such as in, for example, extension of the biological sensor 1 in the longitudinal direction. This can enhance an attachment performance of the biological sensor 1 to the surface of the skin 2. Also, mainly, the capacitance decreases as the relative dielectric constant of the biological sensor 1 decreases. This can suppress generation of a current due to charges at the time of body movements, and noise tends to become smaller. As such, when the relative dielectric constant of the cover member 11 is 2.2 or less regarding the impedance of the biological sensor 1, the contact impedance of the biological sensor 1 can be reduced, and thus the generation of noise can be suppressed. Therefore, the biological sensor 1 can suppress the generation of noise during use, and can be stably attached to the skin 2.

Therefore, the biological sensor 1 can enhance the accuracy of the electrocardiogram waveform measured during measurement of an electrocardiogram as the biological signal, and can enhance the attachment performance to the surface of the skin 2.

No particular limitation is imposed on the evaluation method of the accuracy of the electrocardiogram waveform, and a typical evaluation method can be used. For example, the accuracy of the electrocardiogram waveform can be evaluated by determining a coefficient of determination (RRI coefficient of determination) R² of the interval (RRI) between the highest point R of the QRS complex and the highest point R of the next neighboring QRS complex in an electrocardiogram waveform appearing as the P wave, the QRS complex, and the T wave, as illustrated in FIG. 5.

The cover member 11 of the biological sensor 1 may contain a thermoplastic elastomer. The thermoplastic elastomer has high flexibility and low relative dielectric constant. Thus, the flexibility of the cover member 11 can be enhanced, and the capacitance of the cover member 11 can be lowered. Therefore, the biological sensor 1, which includes the cover member 11 containing the thermoplastic elastomer, can reliably suppress the noise generated during use and stably maintain the attachment performance to the surface of the skin 2.

The biological sensor 1 can use a styrene-based thermoplastic elastomer as the thermoplastic elastomer. The styrene-based thermoplastic elastomer has higher flexibility and a lower relative dielectric constant among other thermoplastic elastomers. Thus, the styrene-based thermoplastic elastomer can further enhance the flexibility of the cover member 11, and can further lower the capacitance of the cover member 11. Therefore, by using the styrene-based thermoplastic elastomer as the thermoplastic elastomer for the cover member 11, the biological sensor 1 can more reliably suppress the noise generated during use and more stably maintain the attachment performance to the surface of the skin 2.

The first layer member 10 of the biological sensor 1 can include the first base 121, the first adhesive layer 122, and the upper adhesive layer 123. Because the first adhesive layer 122 has adhesiveness, the electrode 20 can contact the surface of the skin 2 in a state in which the electrode 20 is attached to the first layer member 10 via the first adhesive layer 122. Therefore, the biological sensor 1 can maintain a state of the electrode 20 stably attached to the skin 2, and can suppress displacement even if body movements occur. This can reduce the contact impedance of the electrode 20 with the surface of the skin 2, and can suppress the generation of noise and more stably attach the electrode 20 to the skin 2. Therefore, the biological sensor 1 can enhance the detection accuracy of biological signals during use, and can maintain the attachment performance to the skin 2.

The biological sensor 1 can include the second adhesive layer 43 on the surface of the second layer member 40 opposite to the first layer member 10. Thus, the second layer member 40 of the biological sensor 1 can be attached to the skin 2 via the second adhesive layer 43. This can further reduce the contact impedance of the electrode 20 with the surface of the skin 2, and can suppress the generation of noise. Therefore, the biological sensor 1 can stably suppress the generation of noise during use, and can maintain the attachment performance to the skin 2.

The biological sensor 1 can form the attachment surface to the skin 2 by the first layer member 10, the electrode 20, and the second layer member 40. Thus, the thickness of the biological sensor 1 can be reduced. Therefore, the biological sensor 1 can be reduced in size, and can reduce the contact impedance with the surface of the skin 2.

As described above, the biological sensor 1 can stably measure biological information from the skin 2 during use for a long time. Thus, the biological sensor 1 can be effectively used as an attachable biological sensor that is attached in use to the skin 2 of a human or the like. For example, the biological sensor 1 exhibits high detection sensitivity of an electrocardiogram when attached to the skin of the living body or the like. Thus, the biological sensor 1 can be successfully used, for example, in a wearable device for health care that requires a high effect of suppressing noise generated in the electrocardiogram.

Although the embodiments have been described above, the above embodiments are merely illustrative, and the present invention is not limited to the above embodiments. The above embodiments can be practiced in various other forms, and various combinations, omissions, substitutions, changes, and the like can be made without departing from the gist of the invention. These embodiments and variations are encompassed in the scope and gist of the invention, and included in the scope equivalent to the inventions recited in the claims.

### EXAMPLES

The embodiments will be described in more detail with reference to Examples and Comparative Examples. However, the embodiments are not limited to these Examples and Comparative Examples.

### Example 1

### [Preparation of Cover Member and Measurement of Physical Properties]

### (Preparation of Cover Member)

### (1) Preparation of Cover Member 1

Thermoplastic elastomer 1 (LIR9826N, obtained from RIKEN TECHNOS CORP.) was used as a base resin, and molded into a predetermined shape, thereby preparing cover member 1.

### (2) Preparation of Cover Member 2

Thermoplastic elastomer 2 (AR-830C, obtained from ARONKASEI CO., LTD.) was used as a base resin, and molded into a predetermined shape, thereby preparing cover member 2.

### (3) Preparation of Cover Member 3

Silicone rubber 1 (CHN-9300-U, obtained from TOPCO TECHNOLOGIES CORP.) and silicone rubber 2 (CHN-9500-U, obtained from TOPCO TECHNOLOGIES CORP.) were mixed at a ratio of 1:1, thereby preparing a mixture having a Shore A hardness of 40. This mixture was used as a base resin, and molded into a predetermined shape, thereby preparing cover member 3.

### (4) Preparation of Cover Member 4

Silicone rubber 3 (CHN-9400-U, obtained from TOPCO TECHNOLOGIES CORP.) and silicone rubber 4 (CHN-9600-U, obtained from TOPCO TECHNOLOGIES CORP.) were mixed at a ratio of 1:1, thereby preparing a mixture having a Shore A hardness of 50. This mixture was used as a base resin, and molded into a predetermined shape, thereby preparing cover member 4.

### (5) Preparation of Cover Member 5

Silicone rubber 2 (CHN-9500-U, obtained from TOPCO TECHNOLOGIES CORP.) and silicone rubber 5 (CHN-9700-U, obtained from TOPCO TECHNOLOGIES CORP.) were mixed at a ratio of 1:1, thereby preparing a mixture having a Shore A hardness of 60. This mixture was used as a base resin, and molded into a predetermined shape, thereby preparing cover member 5.

### (6) Preparation of Cover Member 6

Silicone rubber 4 (CHN-9600-U, obtained from TOPCO TECHNOLOGIES CORP.) and silicone rubber 6 (CHN-9800-U, obtained from TOPCO TECHNOLOGIES CORP.) were mixed at a ratio of 1:1, thereby preparing a mixture having a Shore A hardness of 70. This mixture was used as a base resin, and molded into a predetermined shape, thereby preparing cover member 6.

### (7) Preparation of Cover Member 7

Silicone rubber 6 (CHN-9800-U, obtained from TOPCO TECHNOLOGIES CORP.) was used, thereby preparing a mixture having a Shore A hardness of 80. This mixture was used as a base resin, and molded into a predetermined shape, thereby preparing cover member 7.

### (8) Preparation of Cover Member 8

Thermoplastic elastomer 2 (HYTREL (registered trademark) SB654, obtained from DU PONT-TORAY CO., LTD.) was used as a base resin, and molded into a predetermined shape, thereby preparing cover member 8.

### (Measurement of Physical Properties of Cover Member)

Cover members 1 to 8 were measured for physical properties, i.e., Shore A hardness, tensile modulus, the amount of charge, relative dielectric constant, and adhesive strength.

### (1) Shore A Hardness

The thermoplastic elastomers or the mixtures forming cover members 1 to 8 were each formed into a sheet, thereby preparing sheet samples (length of 60 mm×width of 60 mm×thickness of 10 mm) as test pieces. In accordance with “Rubber, vulcanized or thermoplastic—Determination of hardness Part 3: Durometer method” of JIS K 6253-3:2012, the test pieces (thickness: 10 mm each) of cover members 1 to 8 were each measured using a type A durometer 15 mm from the end of each of the test pieces. The measured values of the test pieces were regarded as the Shore A hardness of each of cover members 1 to 8.

### (2) Tensile Modulus

In accordance with JIS K7161: 2014, cover members 1 to 8 were each cut to prepare rectangular test pieces (samples) each having a size of 30 mm in length 10 mm in width. Both ends of each of the test piece were held between chucks such that the distance between the chucks would be 20 mm. In this state, a tensile test was performed under conditions in which the temperature was normal temperature (23° C.±2° C.) and the tensile speed was 30 mm/min, thereby obtaining a stress-strain curve. The tensile modulus at normal temperature (23° C.±2° C.) was calculated in accordance with the obtained stress-strain curve, i.e., by determining the slopes of the curve at two points at which the strain was 0.05% and 0.25%. A tensile modulus E (unit: MPa) at normal temperature (23° C.±2° C.) was determined by dividing the difference in stress (σ2−σ1) by the difference in strain (ε2−ε1), as presented in Formula (1) below:

$$E = ( σ2 - σ1 ) / ( ε2 - ε1 ) , \qquad ( 1 )$$

where ε1 denotes a value when the strain (unit: %) of each of cover members 1 to 8 is 0.05%, ε2 denotes a value when the strain (unit: %) of each of cover members 1 to 8 is 0.25%, σ1 denotes the stress (unit: MPa) corresponding to ε1, and σ2 denotes the stress (unit: MPa) corresponding to ε2.

### (3) Relative Dielectric Constant

Cover members 1 to 8 were each cut to prepare circular test pieces (samples) each having a diameter of 38 mm. A capacitance (electric capacitance) C of each of the test pieces was measured using an impedance analyzer. The relative dielectric constant, ε/ε₀, was calculated from the measured capacitance C. Specifically, the relative dielectric constant (ε/ε₀) of each of cover members 1 to 8 was calculated by dividing a value C×d, the product of the capacitance C of the test piece and the diameter d of the test piece, by the area S of the test piece and the square of the dielectric constant in vacuum, ε₀, (≈8.85×10−12 F/m), i.e., (C×d/(S×ε₀²)). The relative dielectric constant of the test piece is dependent on the frequency. The frequency of an electrocardiogram is mainly from several hertz through 30 Hz. Therefore, the relative dielectric constant at 5 Hz was calculated.

### (4) Amount of Charge

The amount of charge of each of cover members 1 to 8 was measured using an electrostatic meter (YC-102, obtained from AS ONE Corporation) at an environmental temperature of 23° C.±2° C. and at an environmental humidity of 40%.

### (5) Adhesive Strength

In accordance with JIS Z 0237:2000, the adhesive strength of each of cover members 1 to 8 with respect to an adhesive was measured. First, cover members 1 to 8 were each cut into a size of 10 mm wide and 100 mm long to prepare test pieces. One surface of each of the test pieces was attached to a PET film (50 μm in thickness) to which an adhesive was attached, followed by pressure-bonding performed under pressure-bonding conditions in which a roller of 2 kg was caused to go back and forth, thereby preparing measurement samples. As the adhesive, double-sided adhesive tape having an adhesive layer formed of a silicone resin (ST503, obtained from Nitto Denko Corporation) or double-sided adhesive tape having an adhesive layer formed of an acrylic resin (PKE-20, obtained from Nitto Denko Corporation) was used. After the test piece was pressure-bonded to the PET film, aging was performed in an atmosphere of 23° C. and 50% RH for 30 minutes. After aging, in an atmosphere of 25° C. and 50% RH in accordance with JIS Z 0237, the measurement samples were each peeled off from the PET film at a tensile speed of 300 mm/min and a peeling angle of 180° using a tensile tester (Autograph AG-IS 50 N, obtained from Shimadzu Corporation), thereby measuring an adhesive strength at a peeling angle of 180° (unit: N/10 mm).

### [Preparation of Biological Sensor]

### (Preparation of First Stacked Sheet)

A first adhesive layer was formed by attaching double-sided adhesive tape (obtained from Nitto Denko Corporation, thickness: 60 μm) to the lower surface of porous base 1 formed into a rectangular shape (polyolefin foamed sheet (“FOLEC (registered trademark)”, obtained from INOAC CORPORATION, thickness: 0.5 mm)). The double-sided adhesive tape is double-sided adhesive tape in which an adhesive (acrylic resin) is formed on the surfaces. Subsequently, silicone tape (“ST503 (HC) 60”, obtained from Nitto Denko Corporation, thickness: 60 μm) was attached to the upper surface of the attachment layer to form an upper adhesive layer, thereby preparing a first stacked sheet as a first layer member.

### (Preparation of Second Stacked Sheet)

An adhesive (“PERME-ROLL”, obtained from Nitto Denko Corporation, moisture permeability: 21 g/(m²·day)) was adhered to both surfaces of a base formed into a rectangular shape (PET (“PET-50-SCA1(white)”, obtained from Mitsui & Co. Plastics Ltd.), thickness: 38 μm) to form a lower adhesive layer and a second adhesive layer, thereby preparing a second stacked sheet as a second layer member.

### (Preparation of Biological Sensor)

A sensor portion including a battery and a controller was placed in a center region of the upper surface of the second stacked sheet. Subsequently, a pair of the electrodes were attached to the attachment surface of the first adhesive layer of the first stacked sheet in a state of being held between the first adhesive layer and the second stacked sheet, thereby connecting the electrodes to the interconnects of the sensor portion. Subsequently, the cover member 1 was stacked on the first stacked sheet such that the sensor portion was disposed in a housing space formed by the first stacked sheet and the cover member 1, thereby preparing a biological sensor.

### Example 2 and Comparative Examples 1 to 6

Biological sensors were prepared in the same manner as in Example 1 except for making changes as illustrated in Table 1 differing from Example 1.

### Evaluation of Characteristics of Biological Sensor

The waveform accuracy and the attachment performance of the biological sensor in the Examples and Comparative Examples were measured and evaluated.

### [Waveform Accuracy]

The obtained biological sensors were each attached to skin of a subject for 24 hours, and an electrocardiogram was measured to obtain an electrocardiogram waveform. When there is no noise, an electrocardiogram waveform appears as a P wave, a QRS complex, and a T wave, as illustrated in FIG. 5, and the interval (RRI) between the highest point R of the QRS complex and the highest point R of the next neighboring QRS complex is determined. The obtained electrocardiogram waveform was evaluated in accordance with the following evaluation criteria by determining the RRI coefficient of determination R² using a Lorentz plot. The RRI coefficient of determination R² is a coefficient of determination obtained when a scatter plot of the n^{th} and (n+1)^{th} RRI values is created and linear approximation is performed on this scatter plot. When the RRI coefficient of determination R² is 0.9 or more, it is evaluated that there is substantially no noise and the waveform accuracy is excellent (described as A in Table 1). When the RRI coefficient of determination R² is 0.7 or more and less than 0.9, it is evaluated that there is slight noise but the R wave can be detected, and the waveform accuracy is good (described as B in Table 1). When the RRI coefficient of determination R² is 0.5 or more and less than 0.7, it is evaluated that there is large noise, the R wave is not readily detected, and the waveform accuracy is poor (described as C in Table 1).

### (Evaluation Criteria)

A: The RRI coefficient of determination R² is 0.9 or more.
B: The RRI coefficient of determination R² is 0.7 or more and less than 0.9.
C: The RRI coefficient of determination R² is 0.5 or more and less than 0.7.

### [Attachment Performance]

The attachment performance of the biological sensor was evaluated in accordance with the following evaluation criteria when attaching the biological sensor to a subject for 24 hours and measuring an electrocardiogram.

### (Evaluation Criteria)

A: The biological sensor remains attached to the skin of the subject.
B: At least a part of the biological sensor is peeled off from the skin of the subject.

TABLE 1

| | Cover member | | | | | | | | | |
|---|---|---|---|---|---|---|---|---|---|---|
| | | | Physical properties | | | | | | | |
| | | | | | Relative | | Adhesive strength | | | |
| | | | | Tensile | dielectric | Amount of | [N/10 mm] | | Biological sensor | |
| | | Material | Shore A | modulus | constant | charge | With respect to | With respect to | Waveform | Attachment |
| | Type | Substance | hardness | [MPa] | (@5 Hz) | [kV] | silicone resin | acrylic resin | accuracy | performance |
| Ex. 1 | Cover | Thermoplastic | 40 | 1.5 | 2.2 | 0.4 | 5.3 | 2.50 | A | A |
| | member 1 | elastomer 1 | | | | | | | | |
| Ex. 2 | Cover | Thermoplastic | 35 | 1.0 | 2.2 | −1.0 | 0.5 | 0.05 | B | A |
| | member 2 | elastomer 2 | | | | | | | | |
| Comp. | Cover | Silicone rubber | 40 | 1.2 | 3.4 | −2.0 | 1.7 | 0.03 | B | A |
| Ex. 1 | member 3 | 1:Silicone | | | | | | | | |
| | | rubber 2 = 1:1 | | | | | | | | |
| Comp. | Cover | Silicone rubber | 50 | 1.2 | 3.4 | −2.0 | 1.7 | 0.03 | B | B |
| Ex. 2 | member 4 | 3:Silicone | | | | | | | | |
| | | rubber 4 = 1:1 | | | | | | | | |
| Comp. | Cover | Silicone rubber | 60 | 1.5 | 3.4 | −2.0 | 1.7 | 0.03 | B | B |
| Ex. 3 | member 5 | 2:Silicone | | | | | | | | |
| | | rubber 5 = 1:1 | | | | | | | | |
| Comp. | Cover | Silicone rubber | 70 | 2.8 | 3.4 | −2.0 | 1.7 | 0.03 | A | B |
| Ex. 4 | member 6 | 4:Silicone | | | | | | | | |
| | | rubber 6 = 1:1 | | | | | | | | |
| Comp. | Cover | Silicone rubber | 80 | 7.2 | 3.4 | −2.0 | 1.7 | 0.03 | A | B |
| Ex. 5 | member 7 | 6 | | | | | | | | |
| Comp. | Cover | Thermoplastic | 65 | 8.4 | 5.8 | 0.3 | 4.3 | 3.50 | C | B |
| Ex. 6 | member 8 | elastomer 3 | | | | | | | | |

From Table 1, it was confirmed in Examples 1 and 2 that the biological sensor satisfied the requirements for use in both of the waveform accuracy and the attachment performance. On the other hand, it was confirmed in Comparative Examples 1 to 6 that at least one or more of the waveform accuracy and the attachment performance of the biological sensor did not satisfy the requirements for use.

Therefore, the biological sensor of each of the Examples, in which the tensile modulus and the relative dielectric constant of the cover member were set to be equal to or less than respective predetermined values, was able to suppress the noise generated in the electrocardiogram during the measurement of the electrocardiogram, i.e., stably obtain the electrocardiogram waveform, and also was able to stably maintain the state of attachment to the skin of the subject, i.e., maintain the attachment performance. Therefore, even if the biological sensor according to the present embodiment is attached to the skin of a subject for a long time (e.g., 24 hours), the biological sensor can be effectively used to measure an electrocardiogram continuously for a long time.

Embodiments of the present invention are, for example, as follows.
<1>A biological sensor, including:
a sensor body configured to obtain biological information;
an electrode connected to the sensor body;
a first layer member including a cover member that includes a housing space in which the sensor body is housed, the electrode being disposed on a lower surface of the first layer member; and
a second layer member that is attached to the lower surface of the first layer member so as to expose the electrode and cover the sensor body, in which
a tensile modulus of the cover member is 1.5 MPa or less and a relative dielectric constant of the cover member is 2.2 or less.
<2>The biological sensor according to <1>, in which
the cover member includes a thermoplastic elastomer.
<3>The biological sensor according to <2>, in which
the thermoplastic elastomer is a styrene-based thermoplastic elastomer.
<4>The biological sensor according to any one of <1>to <3>, in which
the first layer member includes
   a first base including a through-hole at a position corresponding to the housing space, and
   a first adhesive layer that is provided at a surface of the first base, the surface of the first base facing a living body, and to which the electrode is attached, and
   an upper adhesive layer that attaches the cover member and the first base to each other.
<5>The biological sensor according to any one of <1>to <4>, in which
the second layer member includes
   a second adhesive layer at a surface opposite to the first layer member.
<6>The biological sensor according to any one of <1>to <5>, in which
an attachment surface to a living body is formed by the first layer member, the electrode, and the second layer member.

The present application claims priority to Japanese Patent Application No. 2022-102032, filed on Jun. 24, 2022 with the Japan Patent Office, and the entire contents of the above application are incorporated herein by reference.

REFERENCE SIGNS LIST

| | |
|---|---|
| 1 | Biological sensor |
| 2 | Skin |
| 10 | First layer member |
| 11 | Cover member |
| 12 | Upper sheet |
| 12a, 121a, 122a | Through-hole |
| 20, 20A, 20B | Electrode |
| 201A, 201B | Facing portion |
| 202A, 202B | Exposed portion |
| 30 | Sensor portion |
| 31 | Flexible substrate |
| 32 | Sensor body |
| 33A | Connection portion |
| 33A, 33B | Connection portion |
| 34 | Battery |
| 40 | Second layer member |
| 41 | Second base |
| 42 | Lower adhesive layer |
| 43 | Second adhesive layer |
| 111 | Projection |
| 111a | Recess |
| 112A, 112B | Flat portion |
| 121 | First base |
| 122 | First adhesive layer |
| 123 | Upper adhesive layer |
| 321 | Part-mounting portion |
| 322 | Battery-mounting portion |
| 331A, 331B | Interconnect |
| 332A, 332B | Terminal |

1. A biological sensor, comprising:
a sensor body configured to obtain biological information;
an electrode connected to the sensor body;
a first layer member including a cover member that includes a housing space in which the sensor body is housed, the electrode being disposed on a lower surface of the first layer member; and
a second layer member that is attached to the lower surface of the first layer member so as to expose the electrode and cover the sensor body, wherein
a tensile modulus of the cover member is 1.5 MPa or less and a relative dielectric constant of the cover member is 2.2 or less.

2. The biological sensor according to claim 1, wherein the cover member includes a thermoplastic elastomer.

3. The biological sensor according to claim 2, wherein the thermoplastic elastomer is a styrene-based thermoplastic elastomer.

4. The biological sensor according to claim 1, wherein
the first layer member includes
a first base including a through-hole at a position corresponding to the housing space, and
a first adhesive layer that is provided at a surface of the first base, the surface of the first base facing a living body, and to which the electrode is attached, and
an upper adhesive layer that attaches the cover member and the first base to each other.

5. The biological sensor according to claim 1, wherein
the second layer member includes
a second adhesive layer at a surface opposite to the first layer member.

6. The biological sensor according to claim 1. wherein
an attachment surface to a living body is formed by the first layer member, the electrode, and the second layer member.

7. The biological sensor according to claim 2, wherein
an attachment surface to a living body is formed by the first layer member, the electrode, and the second layer member.

8. The biological sensor according to claim 3, wherein
an attachment surface to a living body is formed by the first layer member, the electrode, and the second layer member.

9. The biological sensor according to claim 4, wherein
an attachment surface to a living body is formed by the first layer member, the electrode, and the second layer member.

10. The biological sensor according to claim 5, wherein
an attachment surface to a living body is formed by the first layer member, the electrode, and the second layer member.
